(19) United States
(12) Patent Application Publication
(10) Pub. No.: US 2014/0322184 A1
(43) Pub. Date: Oct. 30, 2014

(54) INDUCIBLE EXPRESSION CASSETTE, AND USES THEREOF

(71) Applicants: INSTITUT NATIONAL DE LA RECHERCHE AGRONOMIQUE (INRA), Paris Cedex 07 (FR); CENTRE NATIONAL DE LA RECERCHE SCIENTIFIQUE (CNRS), Paris Cedex 16 (FR)

(72) Inventors: Pierre Farfournoux, Aurieres (FR); Alain Bruhat, Chanonat (FR); Celine Jousse, Saint Amant Tallende (FR); Anne-Catherine Maurin, Aubiere (FR); Julien Averous, Clermont-Ferrand (FR)

(21) Appl. No.: 14/357,160

(22) PCT Filed: Nov. 6, 2012
(86) PCT No.: PCT/EP2012/004610

§ 371 Date: May 8, 2014

(30) Foreign Application Priority Data
Nov. 8, 2011 (FR) 1103392

(51) Int. Cl. A61K 48/00

(52) U.S. Cl. CPC A61K 48/005
USPC 424/93.21; 435/320.1; 514/44 R; 435/366; 435/370; 435/372

(57) ABSTRACT

The invention relates to an expression cassette including a gene of interest under the control of an inducible promoter, characterized in that said inducible promoter includes at least one CARE regulatory sequence (C/EBP-ATF responsive element) and a minimal promoter. The invention also relates to a vector and a host cell, as well as to a pharmaceutical composition including such a cassette, and to the use thereof for treating diseases by gene therapy.

This patent application claims the priority of French patent application No. 11 03392 filed on Nov. 8, 2011, which is incorporated herein by reference.

### FIELD OF THE INVENTION

The present invention relates to an inducible expression cassette including a CARE (C/EBP-ATF Responsive Element) regulatory sequence and the use thereof in the context of gene therapy.

### PRIOR ART

Gene therapy is a therapeutic strategy based on the transfer of a gene or genes into a cell or a host organism. It was first hypothesized in the late 1960s, and the first clinical trial took place in the U.S. twenty years later. Its concept, first considered in the context of genetic diseases, was quickly expanded to the treatment of a large number of other pathologies, such as cancers, infectious diseases, or cardiovascular diseases.

The goal is to deliver gene medicines to the patient, with most considered strategies using vectors in order to convey the therapeutic gene toward its target cell. The first vectors developed were based on a constitutive expression of the gene medicine. In targeted and nontoxic therapy, it soon appeared preferable to limit the expression of the therapeutic gene to given cell types, at a given time, and/or for a given duration. This led to the use of inducible systems. While many inducible systems are now available to a person skilled in the art, the need for novel systems that are more specific, easily controllable and able to be activated, with a low basal expression, along with minimal side effects, remains an obvious one.

### SUMMARY OF THE INVENTION

The invention relates to an expression cassette including a gene of interest that is operationally linked to an inducible promoter, wherein said inducible promoter includes (i) at least one CARE (C/EBP-ATF Responsive Element) regulatory sequence and (ii) a minimal promoter, as well as the use thereof in treating diseases using gene therapy.

The invention additionally relates to a vector including an expression cassette of the invention, a host cell including a cassette, or a vector of the invention.

The invention relates to a pharmaceutical composition including an expression cassette, an expression vector, or a host cell of the invention.

It also covers an expression cassette, a vector, or a host cell of the invention for use in treating diseases using gene therapy.

The invention furthermore extends to a method for treating diseases using gene therapy, wherein said method includes:
(i) A step for administering an expression cassette, a vector, a host cell, or a pharmaceutical composition of the invention to a patient, and
(ii) A step for inducing the expression of the gene of interest included in said expression cassette, said vector, said host cell, or said pharmaceutical composition.

More specifically, said induction is implemented by placing said patient on an amino-acid-deficient diet, preferably involving an essential amino acid.

Finally, the present invention covers a combination including:
A cassette, a vector, a host cell, or a composition of the invention, and
An amino-acid-deficient diet, preferably an essential-amino-acid-deficient diet for simultaneous, separate, or sequential use in treatment using gene therapy.

### DESCRIPTION OF THE DRAWINGS

FIG. 1 shows, in diagram form, the eIF2α/ATF4 signaling pathway, and in particular the GCN2/ATF4 pathway.

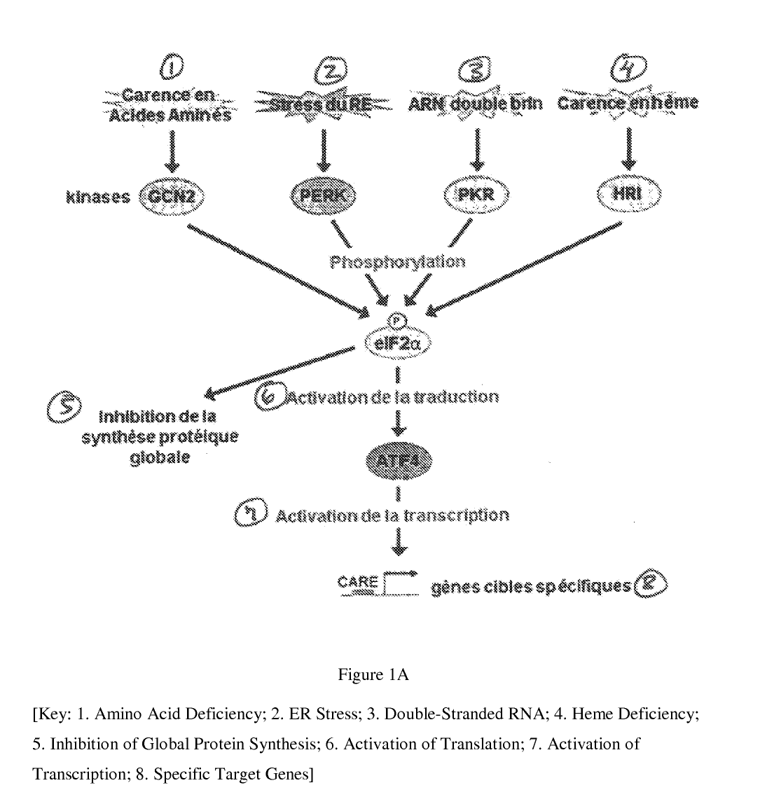

FIG. 1A shows the general signaling pathway.

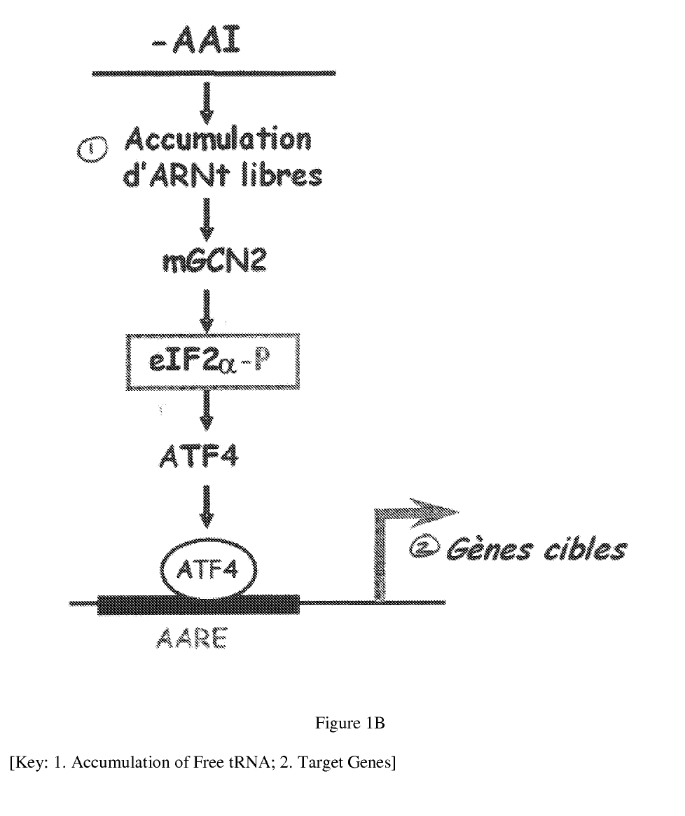

FIG. 1B shows, more specifically, the GCN2/ATF4 signaling pathway. The deficiency of any essential (or indispensable) amino acid activates GCN2 kinase. Following eIF2α phosphorylation, the ATF4 transcription factor is induced. It will bind to the AARE elements located inside the target gene promoter and activate transcription.

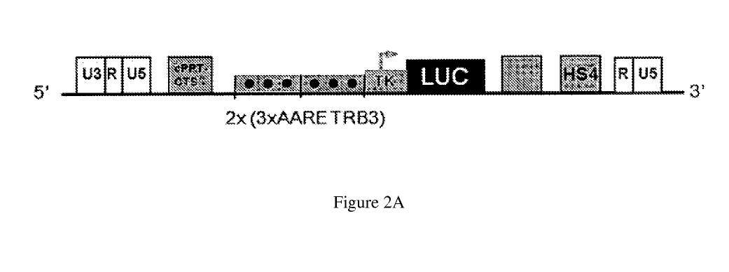

FIG. 2A illustrates a diagram of the 2XAARE-TK-Luc construction (used in the experimental section) inserted into a retroviral expression vector.

FIG. 2B shows the sequence of the 2XAARE-TK-LUC transgene used in the experimental section.

FIG. 3 shows the luciferase activity assay in the tissues of transgenic mice carrying a transgene including a vector of the invention with luciferase as the gene of interest, two copies of the CARE sequence from the TRIB3 gene (AARE sequences), and the thymidine kinase minimal promoter (AARE-LUC mice). At the start of the nutritional experiment, the CARE-LUC mice were fasted for 16 hrs. On the day of the experiment, the mice were fed for four hours, either with the control diet (CTL) or with a leucine-deficient (−leu) diet. After the mice were killed, luciferase activity was assayed in the liver, intestine, pancreas, and brain.

FIG. 4 shows the measurement of luciferase activity after lentivirus was injected into the brain.

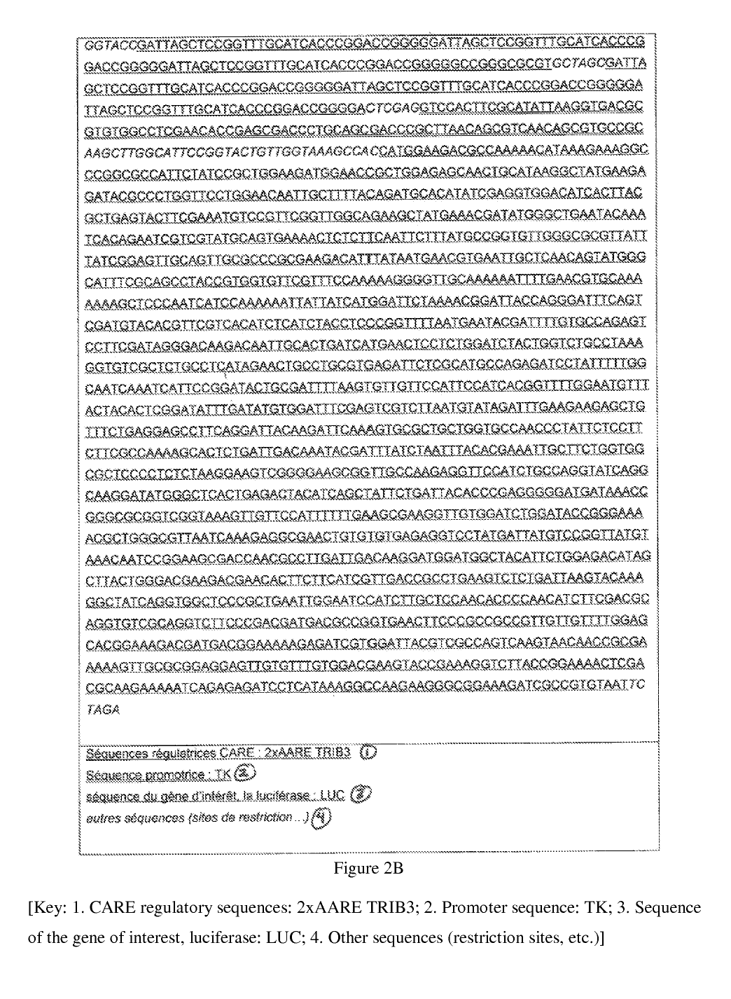

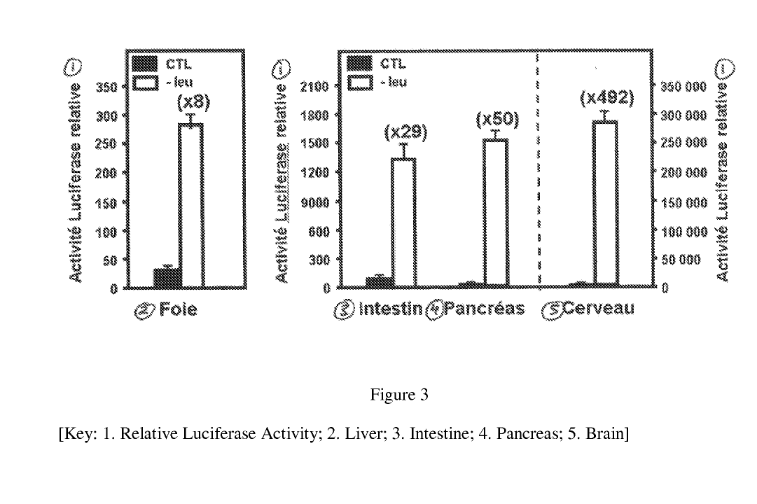

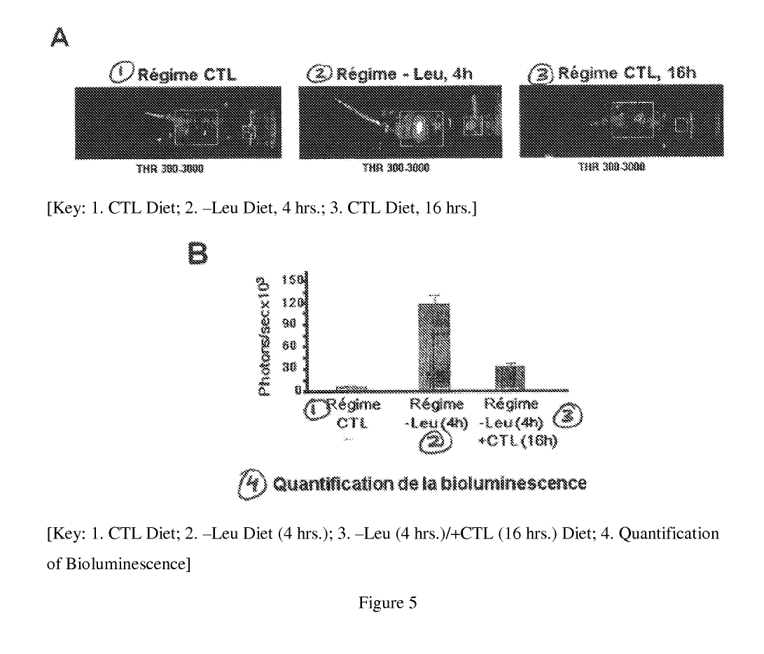

The lentiviral vectors are injected into the hippocampus of the left hemisphere of rats. Two weeks after the injection, the animals are fasted overnight, then fed with a control diet or with a threonine-deficient (−Thr) diet. Six hours after the start of feeding, the animals are killed, and the brains are sampled and dissected. Luciferase activity is measured in the right and left hippocampi and in the remainder of the left hemisphere. Luc activity is then normalized relative to protein content. Two constructions were used: the 2XAARE-TK-LUC construction (described in FIG. 2) and the control TK-LUC construction from which the AARE sequences had been removed.

FIG. 5 shows the reversibility of the induction of the AARE-LUC transgene in mice following consumption of a leucine-deficient diet. (A) Visualization of luciferase activity by bioluminescence imaging (detection limit 300-3000) in a mouse fed successively with (1) a control diet (CTL), (2) a leucine-deficient (−Leu) diet for four hours, and (3) a CTL diet for 16 hours. (B) Quantification of bioluminescence in the boxed areas. The luciferase used has a long half-life, which explains the 16 hours needed to significantly reduce luciferase activity.

### DETAILED DESCRIPTION OF THE INVENTION

The goal of the present invention is an expression cassette including a gene of interest under the control of an inducible promoter, wherein said inducible promoter includes (i) at least one CARE (C/EBP-ATF Responsive Element) regulatory sequence and (ii) a minimal promoter.

The term “expression cassette” refers to an element including specific nucleic acid sequences to be integrated into another nucleic acid molecule or a genome. An expression cassette generally includes one or several genes as well as elements for controlling the expression of said gene(s) (promoter, terminator, etc.).

The term “promoter” is well known to a person skilled in the art, and refers to a DNA region located near a gene to which RNA polymerase binds in order to start transcription.

According to the invention, the promoter is operationally linked to the gene of interest.

Generally, the expression “operationally linked” means that the promoter sequence is positioned relative to the coding sequence of the gene of interest such that transcription is able to start. This means that the promoter is positioned upstream of the coding region, at a distance enabling the latter's expression.

Various types of promoters exist, such as constitutive promoters or inducible promoters. Inducible promoters are promoters whose activity is controlled by specific environmental conditions or by the presence of a specific compound; they therefore make it possible to control the expression of the gene of interest.

The promoters used in the context of the invention may be derived from native genes or from compounds of various elements derived from various natural promoters, or they may include synthetic DNA segments.

A “minimal promoter” is defined for the purposes of this document as a promoter consisting of a transcription start site and functional sequences for binding the transcription start complex (TATA-box) inside a cell or a host organism.

These elements are conventional in the relevant prior art. More specifically, we may mention the minimal promoters of the TK, CMV, and HSP genes (the minimal promoter of the Drosophila heat shock protein gene lacking its activating sequence).

Preferably, the promoter used according to the invention is the thymidine kinase minimal promoter or a derivative thereof. The thymidine kinase minimal promoter corresponds to the −40 to +50 section of the thymidine kinase sequence, with +1 being the transcription start site (Majumder S, DePamphilis M L, Mol Cell Biol, 1994); it is defined by the SEQ ID NO: 1 sequence.

Thymidine kinase minimal promoter sequence
(SEQ ID NO: 1)
GTCCACTTCGCATATTAAGGTGACGCGTGTGGCCTC
GAACACCGAGCGACCCTGCAGCGACCCGCTTAACAG
CGTCAACAGCGTGCCGC

According to the invention, the term “derived from” corresponds to a nucleic acid sequence that has at least 90% identity with a reference sequence, specifically, at least 95% identity and preferably at least 99% identity. The term “percentage of identity between two nucleic acid sequences” refers to the percentage of identical nucleotides between two compared sequences, said percentage being obtained by the best alignment of the entire sequence. The term “best alignment” corresponds to the alignment that yields the highest percentage of identity. It may be obtained by using various algorithms known to a person skilled in the art (GAP, BESTFIT, BLAST P, BLAST N, FASTA, TFASTA, Genetics Computer Group, 575 Science Dr., Madison, Wis. USA).

The promoter may be a eukaryote promoter that must be functional in the host organism.

For the purposes of this document, a “CARE (C/EBP-ATF Responsive Element) regulatory sequence” is defined as any target sequence of the eIF2α/ATF4 signaling pathway that is capable of binding the ATF4 transcription factor. In this pathway, the ATF4 transcription factor (activating transcription factor 4, also referred to as CREB2, TAXREB6, TXREB) binds to these CARE sequences in order to induce or regulate the transcription of target genes in response to a given environmental stress (Kilbert et al., Trends Endocrinol Metab. 2009 November; 20(9): 436-43).

Various CARE sequences have been disclosed, in particular for the following genes: CHOP (C/EBP homologous protein, also called GADD153), ASNS (Asparagine Synthetase), SNAT2 (System A Amino Acid Transporter), ATF3, TRIB3, Cat-1, xCT, HERP, VEGF, and 4E-BP1.

More specifically, in the context of the invention, “Amino Acid Response Elements (AARE)” are defined as CARE regulatory sequences that are targeted by the ATF4 transcription factor in case of amino acid deficiency: these AARE sequences are specific CARE sequences targeted in case of phosphorylation of eIF2α by GCN2 kinase, with this phosphorylation being induced by an amino acid deficiency, preferably an essential amino acid deficiency.

Examples of AARE sequences of the invention include, but are not limited to, the CARE sequences of the CHOP, ASNS, SNAT2, ATF3, and TRIB3 genes.

In a specific embodiment of the invention, the CARE regulatory sequence that is present in the expression cassette includes or consists of a sequence selected from the group including: the CARE sequence of the TRIB3 gene (SEQ ID NO: 2), the CARE sequence of the CHOP gene (SEQ ID NO: 3), the CARE sequence of the ASNS gene (SEQ ID NO: 4), the CARE sequence of the ATF3 gene (SEQ ID NO: 5), and the CARE sequence of the SNAT2 gene (SEQ ID NO: 6), or a derivative thereof.

Preferably, said CARE regulatory sequence that is present in the expression cassette includes or consists of several copies of the SEQ ID NO: 2 sequence, or a derivative thereof.

CARE sequence of the TRIB3 gene
(SEQ ID NO: 2)
CGGTTTGCATCACCCG
CARE sequence of the CHOP gene
(SEQ ID NO: 3)
AACATTGCATCATCCC
AARE sequence of the ASNS gene
(SEQ ID NO: 4)
GAAGTTTCATCATGCC
AARE sequence of the ATF3 gene
(SEQ ID NO: 5)
AGCGTTGCATCACCCC
AARE sequence of the SNAT2 gene
(SEQ ID NO: 6)
GATATTGCATCAGTTT

In a specific embodiment of the invention, the inducible promoter included in the expression cassette of the invention includes one or more copies of a CARE regulatory sequence. More specifically, said promoter includes at least one, at least two, or at least three copies of a CARE regulatory sequence.

More specifically, the inducible promoter included in the expression cassette of the invention includes two copies of a CARE sequence.

Preferably, the inducible promoter included in the expression cassette of the invention includes two copies of the TRIB3 CARE sequence (SEQ ID NO: 2).

In a preferred embodiment, the promoter of the expression cassette of the invention includes two copies of the CARE sequence of the TRIB3 gene (SEQ ID NO: 2) and the thymidine kinase promoter (SEQ ID NO: 1). Preferably, the promoter of the expression cassette of the invention includes or consists of the SEQ ID NO: 7 sequence or a derivative thereof.

SEQ ID NO: 7
GATTAGCTCCGGTTTGCATCACCCGGACCGGGGGATTAGCTCCG
GTTTGCATCACCCGGACCGGGGGATTAGCTCCGGTTTGCATCAC
CCGGACCGGGGGCCGGGCGCGTGCTAGCGATTAGCTCCGGTTTG
CATCACCCGGACCGGGGGATTAGCTCCGGTTTGCATCACCCGGA
CCGGGGGATTAGCTCCGGTTTGCATCACCCGGACCGGGGACTCG
AGGTCCACTTCGCATATTAAGGTGACGCGTGTGGCCTCGAACAC
CGAGCGACCCTGCAGCGACCCGCTTAACAGCGTCAACAGCGTG
CCGC

According to the invention, the CARE regulatory sequence included in the expression cassette is positioned upstream (in position 5′) or downstream (in position 3′) of the minimal promoter. Preferably, it is positioned upstream.

The inducible promoter of the invention, including at least one CARE (C/EBP-ATF Responsive Element) regulatory sequence and a minimal promoter, is preferably inducible by an amino acid deficiency (of one or more amino acids), more preferably by an essential amino acid deficiency.

The gene of interest used in the present invention may derive from a eukaryote organism, a prokaryote, a parasite, or a virus. It may be isolated using any traditional technique in the art, e.g., cloning, PCR, or chemical synthesis. It may be genomic, complementary DNA (cDNA), or mixed-type (minigene). Additionally, it may code for an antisense RNA or an interfering RNA (such as micro-RNA (or miRNA for microRNA), siRNAs (small interfering RNA), dsRNA (double strand RNA), shRNA (short RNA), etc.), and/or a messenger RNA (mRNA) that will subsequently be translated into a polypeptide of interest; the latter may be intracellular, incorporated into the membrane of the host cell, or secreted. This may be a polypeptide such as those found in nature, a portion of the latter, a mutant exhibiting improved or modified biological properties, or a chimeric polypeptide originating from the fusion of sequences having various origins. Moreover, the gene of interest may code for an antisense RNA, a ribozyme, or a polypeptide of interest.

The expression cassette of the invention makes it possible to express, overexpress, or inhibit the expression of a gene of interest. This modulation of the expression of the gene of interest may preferably be controlled by the application or non-application of a diet deficient in one or more amino acids, preferably (an) essential amino acid(s).

Among the usable polypeptides of interest, we may mention more specifically chemokines and cytokines (interferon α, β, or γ, interleukin (IL), specifically IL-2, IL-6, IL-10, or IL-12, tumor necrotizing factor (TNF), colony stimulating factor (GM-, CSF, C-CSF, M-CSF, etc.), MIP-1α, MIP-1β, RANTES, monocyte chemoattractant protein such as MCP-1, etc.), cell receptors (in particular, those recognized by the HIV virus), receptor ligands, coagulation factors (Factor VIII, Factor IX, Factor X, thrombin, C protein, etc.), growth factors (FGF [Fibroblast Growth Factor], VEGF [Vascular Endothelial Growth Factor]), enzymes (kinases, phosphatases, urease, renin, metalloproteinase, NOS [nitric oxide synthetase], SOD, catalase, LCAT [lecithin cholesterol acyl transferase], etc.), enzyme inhibitors (OEI—antitrypsin, antithrombin HI, a viral protease inhibitor, PAI-1 [plasminogen activator inhibitor]), Class-I or Class-II major histocompatibility complex antigens, or polypeptides acting on the expression of the corresponding genes, polypeptides capable of inhibiting a viral, bacterial, or parasitic infection or the development thereof, polypeptides acting positively or negatively on apoptosis (Bax, Bc12, Bc1X, etc.), cytostatic agents (p21, p16, Rb), whole or partial immunoglobulins (Fab, ScFv, etc.), toxins, immunotoxins, apolipoproteins (ApoAI, ApoAIV, ApoE, etc.), angiogenesis inhibitors (angiostatin, endostatin, etc.), markers (β-galactosidase, luciferase, etc.), or any other polypeptide having a therapeutic effect on the targeted condition.

More precisely, in treating a hereditary dysfunction, a functional copy of the defective gene will be used, e.g., a gene coding for Factor VIII or IX in hemophilia A or B, dystrophin (or mini-dystrophin) in Duchenne and Becker muscular dystrophies, insulin in diabetes, and the CFTR (Cystic Fibrosis Transmembrane Conductance Regulator) protein in cystic fibrosis.

For inhibiting the start or progression of tumors or cancers, we preferably use a gene of interest coding for an antisense RNA or an interfering RNA (miRNA, siRNA, dsRNA, shRNA, etc.), a ribozyme, a cytotoxic product (such as the thymidine kinase of Herpes Simplex Virus 1 [TK-HSV-1], ricin, cholera and diphtheria toxins, a product of the FCY1 and FUR1 yeast genes coding for uracyl phosphoribosyl transferase or cytosine desaminase), an immunoglobulin, a cell division or signal transduction inhibitor, an expression product of a tumor suppressor gene (p53, Rb, p′73, DCC, etc.), an immune-system-simulating polypeptide, a tumor-associated antigen (MUC-1, BRCA-1, early or late antigens [E6, E7, L1, L2, etc.] of a human papillomavirus HPV, etc.), optionally combined with a cytokine gene.

An example of a gene of interest that inhibits the expression of a protein would be an shRNA directed against the beta2ACh receptor, which could be used to alleviate tobacco addiction (Maskos et al., Nature 2005).

Finally, as part of anti-HIV therapy, one may use a gene coding for an immunoprotective polypeptide, an antigenic epitope, an antibody, the extracellular domain of the CD4 receptor (sCD4; Traunecker et al., 1988, Nature 331, 84-86), an immunoadhesin (e.g., a CD4-IgG immunoglobulin hybrid, Capon et al., 1989, Nature 33 7, 525-531; Byrn et al., 1990, Nature 344, 667-670), an immunotoxin (e.g., fusion of the 2F5 antibody or of the CD4-2F4 immunoadhesin to angiogenin; Kurachi et al., 1985, Biochemistry 24, 5494-5499), a trans dominant variation (EP 0614980, W095/16780), a cytotoxic product such as one of those mentioned above, or an IFNα or -β.

One of the genes of interest may also be a selection gene enabling the selection or identification of the transfected or transduced cells. We may mention the neo (coding for neomycin phosphotransferase) conferring resistance to the G418 antibiotic, dhfr (Dihydrofolate Reductase), CAT (Chloramphenicol Acetyl Transferase), pac (Puromycin Acetyl Transferase), or gpt (Xanthine Guanine Phosphoribosyl Transferase) genes. Generally speaking, the selection genes are known in the art.

Moreover, the expression cassette of the invention may include additional elements that improve its expression or maintenance in the host cell (replication origins, integration elements in the cell genome, intronic sequences, poly A transcription termination sequences, tripartite leaders, etc.). These elements are known in the art.

Additionally, the gene of interest may also comprise, upstream of the coding region, a sequence coding for a signal peptide enabling its secretion from the host cell. The signal peptide may be that of the gene in question or heterologous (originating from any secreted or synthetic gene).

Finally, the gene of interest contains a terminator.

In a specific embodiment of the invention, the expression cassette of the invention also includes the 5′UTR sequence of the ATF4 gene (SEQ ID NO: 8, Vattem et al., PNAS, 2004; Lu et al., J. Cell. Biol. 2004) or a similar sequence, upstream of the cassette's translational start site.

According to the invention, “a similar sequence” means a 5′UTR sequence of a gene having the same translational regulation as ATF4. Examples of genes having a similar 5′ UTR sequence include, but are not limited to: GADD34 (Lee et al., J Biol Chem, 2009), ATFS (Zhou et al., J Biol Chem, 2008), and BACE1 (Zhou et al, Mol Cell Biol, 2006).

SEQ ID NO: 8
TTTCTGCTTGCTGCTGTCTGCCGGTTTAAGTTGTGTGCTCGGGTGTCC
CTTTCCTCTTCCCCTCCCGCAGGGCTTGCGGCCACCATGGCGTATTAG
AGGCAGCAGTGCCTGCGGCAGCGTTGGCCTTTGCAGCGGCGGCAGCA
GCACCAGGCTCTGCAGCGGCAACCCCCACCGGCCTAAGCCATGGCGC
TCTTCACGAAATCCAGCAGCAGTGTTGCTGTAACGGACAAAGATACC
TTCGAGTTAAGCACATTCCTCGAATCCAGCAAAGCCCCACAACATGA
CCGAGATGAGCTTCCTGAA

Another goal of the invention is a vector including an expression cassette according to the invention.

According to the invention, this may be a synthetic vector (cationic lipids, polymer liposomes, etc.), a plasmid, or a viral vector.

If desired, it may be combined with one or more substances that improve the vector's transfection efficacy and/or stability. These substances are widely documented in the literature available to a person skilled in the art (see, e.g., Felgner et al., 1987, Proc. West. Pharmacol. Soc. 32, 115-121; Hodgson and Solaiman, 1996, Nature Biotechnology 14, 339-342; Remy et al., 1994, Bioconjugate Chemistry 5, 647-654). By way of nonlimiting illustration, they may be polymers, cationic lipids, liposomes, nuclear proteins, or neutral lipids. These substances may be used alone or in combination. One possible combination is a recombinant plasmid vector combined with cationic lipids (DOGS, DC-CHOL, spermine-chol, spermidine-chol, etc.) and neutral lipids (DOPE).

A wide selection of plasmids can be used in the context of the present invention. They may be cloning and/or expression vectors. In general, they are known in the art, and a number of them are commercially available, but it is also possible to construct or modify them using genetic manipulation techniques. By way of examples, we may mention the plasmids derived from pBR322 (Gibco BRL), pUC (Gibco BRL), pBluescript (Stratagène), pREP4, pCEP4 (Invitrogene), or p Poly (Lathe et al., 1987, Gene 57, 193-201). Preferably, a plasmid implemented in the present invention contains a replication origin that ensures the start of replication in a producer cell and/or a host cell (for example, the CoIEI origin will be used for a plasmid to be produced in E. coli, and the oriP/EBNAI system will be used if it is to be self-replicative in a mammal host cell, Lupton and Levine, 1985, Mol. Cell. Biol. 5, 2533-2542; Yates et al., Nature 313, 812-815). It may also include a selection gene for selecting or identifying the transfected cells (complementation of an auxotrophic mutation, gene coding for resistance to an antibiotic, etc.). It may also include additional elements that improve its maintenance and/or its stability in a given cell (a cer sequence that encourages monomer maintenance of a plasmid, integration sequences in the cell genome).

When a viral vector is involved, it may be a vector derived from an adenovirus, a lentivirus, a retrovirus, an adenovirus-associated virus (AAV), a herpes virus, an alphavirus, a parvovirus, a poxvirus (fowlpox, canarypox, vaccinia viruses, in particular of the MVA (Modified Virus Ankara) or Copenhagen strains, etc.) or a foamy virus. Preferably, a nonreplicative and, optionally, nonintegrative vector will be used. Retroviruses have the property of infecting and becoming predominantly integrated into dividing cells and are therefore especially well-suited to an application in anticancer therapy. A suitable retroviral vector for the implementation of the present invention comprises LTR (Long Terminal Repeat) terminal sequences and an encapsidation region. It may derive from a retrovirus of any origin (murine, primate, feline, human, etc.) and, in particular, may derive from a retrovirus selected from the group including MoMuLV (Moloney Murine Leukemia Virus), MVS (Murine Sarcoma Virus), or Friend Murine Retrovirus (Fb29). It is propagated in an encapsidation line that is able to provide in trans the gag, pol, and/or env viral polypeptides needed for the constitution of a viral particle. These types of lines are described in the literature (PA317, Psi CRIP GP+Am-12, etc.). The retroviral vector of the invention may comprise modifications, in particular at the LTRs (replacement of the promoter region by a eukaryote promoter) or at the encapsidation region (replacement by a heterologous encapsidation region, e.g., of the VL30 type) (see French patent applications 94 08300 and 97 05203).

In a preferred embodiment of the invention, the vector is a lentiviral vector, an adenoviral vector, or a vector derived from an adenovirus-associated virus (AAV).

According to an advantageous embodiment, the viral vector used according to the invention may be in the form of a DNA vector or of an infectious viral particle.

The present invention also relates to a host cell including a vector or a cassette according to the invention.

For the purposes of the present invention, a cell of this type is composed of any cell that is transfectable by a vector as described above.

Specifically, a mammal cell, preferably a human cell, may be used. It may be a primary or tumor cell of any origin, in particular hematopoietic (totipotent stem cell, leukocyte, lymphocyte, monocyte or macrophage, etc.), muscular (satellite cell, myocyte, myoblast, smooth muscle, etc.), cardiac, pulmonary, tracheal, hepatic, epithelial, or fibroblast, but also stem cells.

Another goal of the present invention is a pharmaceutical composition including an expression cassette of the invention, an expression vector of the invention, or a host cell of the invention.

In a preferred embodiment of the invention, this type of pharmaceutical composition is intended for use in the treatment and/or prevention of diseases using gene therapy.

A composition of the invention is more specifically intended for use in the preventive or curative treatment of diseases using gene therapy (including immunotherapy) and applies more specifically to proliferative diseases (cancers, tumors, dysplasias, etc.), infectious—specifically, viral—diseases (induced, e.g., by the Hepatitis B or C viruses, HIV [Human Immunodeficiency Virus], herpes, retroviruses, etc.), genetic diseases (cystic fibrosis, myopathies, hemophilias, diabetes, etc.), cardiovascular diseases (restenosis, ischemia, dyslipidemia, etc.), or neurological diseases (psychiatric diseases, neurodegenerative diseases such as Parkinson's or Alzheimer's, addictions [e.g., to tobacco, alcohol, or drugs], epilepsy, etc.).

A composition according to the invention may be manufactured following conventional practices for local, parenteral, or digestive administration, but also by stereotaxy. Specifically, a therapeutically effective quantity of the therapeutic or prophylactic agent is combined with a pharmaceutically acceptable support. There are multiple routes of administration. We may mention, e.g., the intragastric, subcutaneous, intracardiac, intramuscular, intravenous, intraarterial, intraperitoneal, intratumoral, intranasal, intrapulmonary, or intratracheal routes. For these final three embodiments, spray administration or instillation is advantageous.

Administration can take place in a single dose or in a dose that is repeated one or more times after a certain time interval has elapsed. The administration route and appropriate doses vary based on various parameters, e.g., involving the individual, the pathology, the gene of interest to be transferred, or the administration route. When a vector is used, doses including from 0.01 to 100 mg DNA, preferably 0.05 to 10 mg and, especially preferred, from 0.5 to 5 mg may be implemented.

The formulation may also include a pharmaceutically acceptable diluent, adjuvant, or excipient, as well as solubilizers, stabilizers, and preservatives. A preferred composition is in injectable form. It may be formulated in an aqueous, saline (phosphate, monosodium, disodium, magnesium, potassium, etc.), or isotonic solution.

It may be presented in single-dose or multiple-dose formats, in liquid or dry form (powder, lyophilisat, etc.) that can be reconstituted extemporaneously using an appropriate diluent.

The present invention also relates to the therapeutic or prophylactic use of an expression cassette, a vector, or a host cell of the invention, for the preparation of a drug for the transfer and expression of a gene of interest (included in said expression cassette, said vector, or said host cell) in a cell or a host organism. In particular, the expression cassette, vector, or host cell of the invention is intended for treating the human or animal body using gene therapy.

The goal of the invention is therefore an expression cassette, a vector, or a host cell of the invention for use in treating diseases using gene therapy.

According to a first option, the drug may be administered directly in vivo (e.g., by intravenous injection, into an accessible tumor, into the lungs using a spray, into the vascular system using an appropriate probe, or by stereotaxy into the brain). The ex vivo approach may also be used, which consists of sampling cells from the patient (bone marrow stem cells, peripheral blood lymphocytes, muscle cells, etc.), transfecting them in vitro using techniques known in the art, and re-administering them to the patient after an optional amplification step.

The prevention and treatment of many pathologies may be envisaged. A preferred use consists of treating or preventing cancers, tumors, and diseases resulting from undesired cell proliferation. Among possible applications, we may mention cancers of the breast, uterus (in particular, those induced by HPV papillomaviruses), prostate, lung, bladder, liver, colon, pancreas, stomach, esophagus, larynx, central nervous system, and blood (lymphomas, leukemia, etc.). It is also useful in cardiovascular diseases, e.g., for inhibiting or delaying the proliferation of smooth muscle cells of the vascular wall (restenosis). Moreover, with regard to infectious diseases, it may be used in treating AIDS (Acquired Immune Deficiency Syndrome). Lastly, it is particularly appropriate for the treatment of neurological diseases (psychiatric, neurodegenerative diseases, addictions, etc.).

The invention also extends to a method for treating diseases using gene therapy, wherein said method includes:
(iii) A step for administering an expression cassette, a vector, a host cell, or a pharmaceutical composition of the invention to a patient, and
(iv) A step for inducing the expression of the gene of interest included in said expression cassette, said vector, said host cell, or said pharmaceutical composition.

In the context of the present invention, the term “patient” refers to a mammal, preferably a human, suffering from a pathology that can be treated by gene therapy. These pathologies are known in the art, and include cancers, infectious diseases, cardiovascular diseases, and genetic diseases. Examples are provided in the present description.

The administration step is performed using methods known in the art.

The induction step is performed by causing said patient to experience an environmental stimulus that induces the phosphorylation of eIF2α (and hence the activation of the eIF2α/ATF4 signaling pathway).

In an embodiment of the invention, the stimulus experienced by the patient is an amino acid deficiency, preferably an essential amino acid deficiency.

In this embodiment, the expression cassette (included or not included in a vector or host cell) of the invention more specifically includes the CARE regulatory sequence of a gene selected from the group including TRIB3, CHOP, ASNS, ATF3, and SNAT2, or a sequence derived therefrom. Preferably, the promoter used is the promoter defined by the SEQ ID NO: 7 sequence.

In another embodiment of the invention, the stimulus experienced by the patient is the induction of a viral infection or the administration of a molecule mimicking a viral infection. The eIF2α/ATF4 pathway is activated during a viral infection by the presence of double-stranded RNA produced by the virus, by the cytokines and interferon, or by PACT protein and heparin. Hence, a viral infection may be mimicked by one of these elements.

In another embodiment, the stimulus experienced by the patient is the induction of endoplasmic reticulum stress. This type of stress may be induced by a number of drugs, such as the anticancer drug bortezomyb, tunicamycin, dithiothreitol, thapsigargin, or brefeldin A, or by a lipopolysaccharide injection.

In another embodiment, the stimulus experienced by the patient is the induction of a heme deficiency.

In another embodiment, the stimulus experienced by the patient is a lipid-heavy diet.

In another embodiment, the stimulus experienced by the patient is heat shock or osmotic shock.

In a preferred embodiment of the invention, the patient is placed on a diet deficient in an essential amino acid (also referred to as an indispensable amino acid). The essential amino acids are phenylalanine, leucine, methionine, lysine, isoleucine, valine, threonine, tryptophan, and histidine.

For the purposes of this document, an “amino-acid-deficient diet” is defined as any means or any composition for enforcing an amino acid deficiency in a patient as described above. Preferably, the patient is caused to have a deficiency in an essential amino acid. This may involve compositions including a cocktail of free amino acids in which an amino acid, preferably an essential amino acid, is absent.

This type of diet will be preferably administered after a short-term fast, and may be given in the form of a cocktail of free amino acids or a full meal in which the proteins are replaced by a mixture of free amino acids. The essential amino acid blood depletion occurs very quickly (a few minutes after consuming the deficient meal) and may be stopped very quickly by administering the lacking essential amino acid. The deficiency of any of the nine indispensable amino acids is functional. The selection of the amino acid (or amino acids) to be withdrawn from the patient's food may be made by a person skilled in the art, in order to minimize treatment-related side effects, and taking into account the ease with which deficient food may be prepared.

It is moreover possible, for medium- or long-term treatment, to alternate the deficiency in various essential amino acids. Since a single meal deficient in a single amino acid, preferably essential, is not toxic, the use of a cassette of the invention offers a unique advantage relative to other inducible systems that are not usable in human medicine.

In a specific embodiment, the patient is placed on successive diets that are deficient in one amino acid, with each being deficient in a different amino acid (e.g., a leucine-deficient diet, followed by a valine-deficient diet, then a lysine-deficient diet, etc.). This makes it possible to maintain a deficiency enabling the induction of the expression of the gene of interest of the invention without inflicting a lengthy deficiency in one amino acid upon the patient, which might be harmful.

It is possible to place the patient on a diet that is deficient in one or more amino acids.

The use of a cassette of the invention for inducing the expression of the gene via an amino acid deficiency makes it possible to precisely control the start and duration of the induction period for the expression of the gene of interest. Moreover, the post-induction response, as well as the extinction of the expression of the gene of interest (by administering the lacking amino acid), are rapid. Additionally, the expression cassette of the invention leads to a low basal expression level and a high activation level.

In another embodiment of the invention, the patient is administered an amino-acid-consuming enzyme.

An example of an amino-acid-consuming enzyme is asparaginase. Said amino-acid-consuming enzyme is preferably administered intravenously. This type of administration is well known to a person skilled in the art (Pieters R. et al., Cancer, 2011; Patil S. et al., Cancer Treat Rev, 2011).

In another specific embodiment, the cassette of the invention is used in pathologies wherein the eIF2alpha/ATF4 pathway is activated, such as cancers (Ye et al., EMBO J, 2010) or epilepsy (Carnevalli et al., Biochem J, 2006). In this specific embodiment, the step for inducing the expression of the gene of interest (ii) of the method of the invention is no longer necessary.

In this context, the invention relates to a method for treating pathologies wherein the eIF2alpha/ATF4 pathway is activated using gene therapy, with said method including a step for administering an expression cassette, a vector, a host cell, or a pharmaceutical composition of the invention to a patient.

Such pathologies include, in particular, cancers and epilepsy.

In a preferred embodiment, the invention relates to a method for treating diseases using gene therapy, with said method including:
(i) A step for administering an expression cassette, a vector, a host cell, or a pharmaceutical composition of the invention to a patient, and
(ii) A step for inducing the expression of the gene of interest included in said expression cassette, said vector, said host cell, or said pharmaceutical composition, wherein said induction is (a) implemented by placing said patient on a diet that is deficient in an essential amino acid and (b) is simultaneous with, separate from, or sequential to Step (i).

The patient may be placed on successive amino-acid-deficient diets, with each being deficient in a different amino acid as described above. It is also possible to place the patient on a diet that is deficient in one or more amino acids, preferably a diet that is deficient in one or more essential amino acids.

Finally, the present invention covers a combination including:
A cassette, a vector, a host cell, or a composition of the invention, and
A diet that is deficient in amino acids, preferably a diet that is deficient in essential amino acids
   for simultaneous, separate, or sequential use in treating diseases using gene therapy, as described above.

### EXAMPLES

Other features of the invention will emerge in the following examples; however, these examples in no way limit the scope of the invention.

Material and Methods

Creating the AARE-TK-LUC Transgene.

The 2XAARE TRIB3-TK-LUC construction shown in FIG. 2 was obtained by subcloning a double-stranded oligonucleotide containing two copies of the CARE/AARE sequence of TRIB3 (−7131 to −7033) at the MluI-Xhol site of the TATA-TK-LUC plasmid construction containing the coding sequence of the luciferase gene originating from pGL3 basic (Promega). The construction was sequenced, and then the leucine deficiency response was tested in transient transfection in HepG2 human cells (liver hepatoma) and in murine cells (MEF: Mouse Embryonic Fibroblasts).

The DNA fragment corresponding to the sequences of the 2XARRE-TK-LUC transgene was then cloned at the BamHi/XbaI sites of the pRRL.PPT.SF.GFPpre 1xHS4 lentiviral vector (Schambach A, Maetzig T, Loew R, Baum C. Mol. Ther. 2007 June; 15(6): 1167-73) containing the chicken β-globin 5′HS4 “insulator” sequences. In order to verify the inducibility of the AARE-LUC transgene via the eIF2α/ATF4 signaling pathway, HeLa cells were infected by these lentiviral particles. The results show that the LUC transgene, stably integrated into HeLa cells, is induced by a decrease in the leucine concentration in dose-dependent fashion, on the order of those observed in mouse plasma (30 to 70 μM) and by various concentrations of tunicamycin, an agent that induces endoplasmic reticulum stress.

Generation of Transgenic Mice.

In order to generate the AARE-TK-LUC transgenic mice, the lentiviral particles were microinjected into the perivitelline space of an oocyte at the one-cell stage originating from C5BL/6JxDB/2J mice (Charles River, Wilmington, Mass.). This lentiviral germ line integration yielded over 50% of founders carrying the transgene. Fifteen independent lines were thereby obtained, but only five founders had at least one copy of the integrated transgene. F1 offspring were obtained for these five selected F0 founder mice.

Luciferase Activity Assay in Tissue Extracts.

Luciferase activity in the cell or tissue extracts was measured by using a commercial kit (YELEN, Ensue La Redonne, France). The relative luciferase activity corresponds to the ratio between luciferase activity and protein quantity.

Measurement of Bioluminescence by Imaging.

Luciferase activity was visualized on the living AARE-LUC mice with a NightOWL II LB 983 NC100 in vivo imaging unit (Berthold Technologies, Bad Wildbad, Germany). This system uses a slow-scan ultrasensitive CCD camera cooled using the Peltier effect, equipped with a 25 mm/0.95 lens located in a sealed, heat-controlled darkroom, enabling the detection of very low levels of light emitted by a cell expressing a tracer gene such as luciferase. It is also equipped with an integrated gas anesthesia system. For bioluminescence detection, the mice were anesthetized with isoflurane (induction 5% isoflurane, maintenance at 2% in 70% air—30% oxygen) and received an intraperitoneal injection of an aqueous luciferin solution (Caliper LIFE SCIENCES, 150 mg/kg), 10 minutes prior to measuring photon emission (2×4 min. of integration, 8×8 pixel binning), in order to obtain a uniform biodistribution of the substrate. The bioluminescence images are presented as pseudo-color images superimposed onto a photo acquired prior to the bioluminescence image in grayscale, using the WinLight software program (Berthold Technologies). The intensity of the bioluminescent signal is then quantified at the regions of interest using the WinLight software program and expressed in number of photons per second.

Results

Measurement of Luciferase Activity in Tissues.

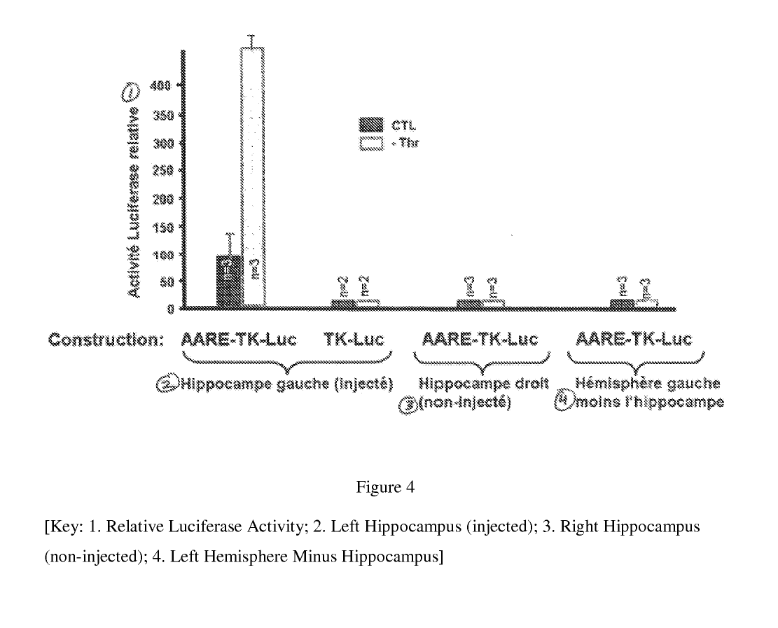

First, the evaluation of the inducibility of the AARE-TK-LUC transgene to leucine deficiency was performed by measuring luciferase activity in the various tissues from the various transgenic lines. Mice of approximately two months of age were habituated, over seven days, to a control (CTL) diet containing all of the indispensable amino acids. Next, after being fasted for 16 hours, they were fed either the CTL diet or a leucine-deficient (−Leu) diet, and then killed so that samples of various tissues could be taken. The luciferase activity analysis shows that consuming a leucine-free diet causes a strong induction of LUC activity in the liver, intestine, pancreas, and brain (FIG. 3). This expression profile of the LUC gene was confirmed by measuring the level of LUC mRNA.

Measurement of Bioluminescence in Living Mice.

Next, the induction of the luciferase gene expression by the leucine deficiency corresponding to the activation of the eIF2α/ATF4 signaling pathway was visualized by bioluminescence imaging on living animals. As before, the mice were first acclimatized with a CTL diet for seven days, and then two types of experiments were conducted:
In the first case, the mice were fasted for 16 hours. On the day of the experiment, they were fed either the CTL diet or a leucine-deficient (−leu) diet for four hours. After the mice were killed, luciferase activity was assayed in various tissues (FIG. 3).
In the second case, mice that had been fasted for 16 hours were fed a leucine-deficient diet for four hours, and then fed a control diet. This experiment measured the reversibility of the transgene's expression (FIG. 5). In this case, the bioluminescence originating from the luciferase was measured before and after the −leu diet was consumed. Photon counting was performed at the abdomen. The results obtained with the ventral side clearly show a strong increase in luminescence in the abdominal region four hours after the start of the meal. We see a considerable decrease in luminescence when the mice are fed a control diet again.

1-15. (canceled)

16. An expression cassette including a gene of interest operationally linked to an inducible promoter, wherein said inducible promoter includes (i) at least one CARE (C/EBP-ATF Responsive Element) regulatory sequence and (ii) a minimal promoter.

17. An expression cassette as claimed in claim 1, wherein the minimal promoter is the thymidine kinase minimal promoter defined by the SEQ ID NO: 1 sequence.

18. An expression cassette as claimed in claim 1, wherein the inducible promoter includes at least one copy of a CARE regulatory sequence selected from the group consisting of the SEQ ID NO: 2-6 sequences.

19. An expression cassette as claimed in claim 1, wherein the inducible promoter includes at least two copies of a CARE regulatory sequence.

20. An expression cassette as claimed in claim 1, wherein the inducible promoter includes at least two copies of a CARE regulatory sequence selected from the group consisting of the SEQ ID NO: 2-6 sequences.

21. An expression cassette as claimed in claim 1, wherein the inducible promoter is the promoter of SEQ ID NO: 7.

22. An expression cassette as claimed in claim 1, wherein said inducible promoter is inducible by an amino acid deficiency.

23. An expression cassette as claimed in claim 1, wherein said inducible promoter is inducible by an essential amino acid deficiency.

24. An expression cassette as claimed in claim 1, wherein said inducible promoter includes an AARE sequence.

25. An expression cassette as claimed in claim 24, wherein the inducible promoter includes the AARE sequences of the CHOP, ASNS, SNAT2, ATF3 or TRIB3 genes.

26. A vector including an expression cassette as claimed in claim 1.

27. A vector as claimed in claim 26, wherein said vector is a lentiviral vector, an adenoviral vector, or a vector derived from an adenovirus-associated virus.

28. A host cell including an expression cassette as claimed in claim 1.

29. A pharmaceutical composition including an expression cassette as claimed in claim 1 or a vector or host cell comprising said expression cassette.

30. A method for treating diseases using gene therapy which comprises administration, to a patient in need of said treatment, the pharmaceutical composition as claimed in claim 29.

31. A method as claimed in claim 30 for the treatment of proliferative diseases, infectious diseases, genetic diseases, neurological diseases, or cardiovascular diseases.

32. A method as claimed in claim 30 for treatment of cancer or viral infection.

33. A method as claimed in claim 30 further comprising simultaneous, separate or sequential application of an amino-acid-deficient diet to said patient.

34. A method as claimed in claim 30, wherein said inducible promoter of the expression cassette includes an AARE sequence.

35. A combination including:
an expression cassette as claimed in claim 1 or a vector, host cell or pharmaceutical composition comprising said expression cassette, and
an amino-acid-deficient diet for simultaneous, separate, or sequential use in treating diseases using gene therapy.


SEQUENCE LISTING (8 sequences)

SEQ ID NO: 1 - DNA, 89 bp, Human Herpes Virus type 1
gtccacttcgcatattaaggtgacgcgtgtggcctcgaacaccgagcgaccctgcagcgacccgcttaacagcgtcaacagcgtgccgc

SEQ ID NO: 2 - DNA, 16 bp, Homo sapiens
cggtttgcatcacccg

SEQ ID NO: 3 - DNA, 16 bp, Homo sapiens
aacattgcatcatccc

SEQ ID NO: 4 - DNA, 16 bp, Homo sapiens
gaagtttcatcatgcc

SEQ ID NO: 5 - DNA, 16 bp, Homo sapiens
agcgttgcatcacccc

SEQ ID NO: 6 - DNA, 16 bp, Homo sapiens
gatattgcatcagttt

SEQ ID NO: 7 - DNA, 311 bp, Artificial Sequence
  Transgene
gattagctccggtttgcatcacccggaccgggggattagctccggtttgcatcacccggaccgggggattagctccggtttgcatcacccggaccgggggccgggcgcgtgctagcgattagctccggtttgcatcacccggaccgggggattagctccggtttgcatcacccggaccgggggattagctccggtttgcatcacccggaccggggactcgaggtccacttcgcatattaaggtgacgcgtgtggcctcgaacaccgagcgaccctgcagcgacccgcttaacagcgtcaacagcgtgccgc

SEQ ID NO: 8 - DNA, 300 bp, Homo sapiens
tttctgcttgctgtctgccggtttaagttgtgtgctcgggtgtccctttcctcttcccctcccgcagggcttgcggccaccatggcgtattagaggcagcagtgcctgcggcagcgttggcctttgcagcggcggcagcagcaccaggctctgcagcggcaacccccaccggcctaagccatggcgctcttcacgaaatccagcagcagtgttgctgtaacggacaaagataccttcgagttaagcacattcctcgaatccagcaaagccccacaacatgaccgagatgagcttcctgaa